(19) United States
(12) Patent Application Publication
(10) Pub. No.: US 2018/0192661 A1
(43) Pub. Date: Jul. 12, 2018

(54) METHODS OF USING FUNGI TO ACIDIFY MILK

(71) Applicant: CARGILL, INCORPORATED, Wayzata, MN (US)

(72) Inventor: Brian Jason BRAZEAU, Excelstor, MN (US)

(73) Assignee: Cargill, Incorprated, Wayzata, MN (US)

(21) Appl. No.: 15/107,227

(22) PCT Filed: Feb. 5, 2015
(86) PCT No.: PCT/US15/14573

§ 371 Date: Jun. 22, 2016

Related U.S. Application Data
(60) Provisional application No. 61/936,538, filed on Feb. 6, 2014.

(51) Int. Cl. A23C 9/12; C12N 1/16; A23C 9/127; A23C 19/032

(52) U.S. Cl. CPC A23C 9/1203; A23C 19/0326; A23C 9/127; C12N 1/16

(57) ABSTRACT

Disclosed herein are methods of acidifying milk comprising adding at least one microorganism belonging to the Kingdom Fungi to milk. In certain embodiments, the milk is acidified to a pH of less than about 6. Also disclosed are methods of making a dairy product comprising milk, wherein the milk is acidified using at least one microorganism belonging to the Kingdom Fungi to acidify milk. Further disclosed are dairy products, such as cheese, made by acidifying milk using at least one microorganism belonging to the Kingdom Fungi.

### CROSS-REFERENCE TO RELATED APPLICATIONS

This application claims the benefit of the U.S. Patent Application Ser. No. 61/936,538 filed Feb. 6, 2014, entitled METHODS OF USING FUNGI TO ACIDIFY MILK, which application is hereby incorporated by reference herein in its entirety.

### DESCRIPTION

### Technical Field

The present disclosure relates to methods of acidifying milk comprising adding at least one microorganism belonging to the Kingdom Fungi to milk. In certain embodiments, the milk is acidified to a pH of less than about 6. Further disclosed are methods of making a dairy product, such as cheese, using at least one microorganism belonging to the Kingdom Fungi. Dairy products, such as cheese, made by acidifying milk using at least one microorganism belonging to the Kingdom Fungi are also disclosed herein.

### Background

There are a number of processing steps involved in producing dairy products. By way of example, in the production of various kinds of cheese, the protein and fat ratio of the milk may first be optimized in order to make good quality dairy products with high yield.

Next, the milk may be pasteurized or heat treated. Pasteurization or heat treatment may reduce the number of spoilage organisms and/or improve the environment for starter cultures to grow.

After pasteurization, the milk is cooled and then may be inoculated with at least one starter culture, such as lactic acid starter bacteria. Lactic acid starter bacteria are typically added during the procedure in order to produce lactic acid and begin the fermentation process. These lactic acid starter bacteria are used early in the process to assist with coagulation, e.g. by lowering the pH of the mixture. The metabolism of the lactic acid starter bacteria may also contribute to desirable flavor compounds, and/or help prevent the growth of spoilage organisms.

After the milk has been inoculated, rennet may be added to form curd in the milk. Rennet is an enzyme that acts on milk proteins to form the curd. After the rennet is added, the curd is not disturbed for a period of time, such as about 30 minutes, so that a firm coagulum forms. Next, the curd may be cut and heated, which may help to separate the whey from the curd. After the heating step, the whey may be drained from the cheese, and the curd may form a mat. The curd mats may then be cut into sections and piled on top of each other and flipped periodically. Flipping the curd mats may help to expel more whey and/or allow the fermentation to continue until a desired pH is reached, for example a pH ranging from about 5.1 to about 5.5. At this point, the curd mats may “knit” together and form a tighter matted structure. The curd mats may then be milled into smaller pieces.

For the manufacture of certain types of cheese, such as, for example, cheddar cheeses, the milled curd pieces may be put back into the cheese vat and salted, for example by sprinkling dry salt on the curd. In other cheese varieties, the curd may be formed into loaves, and the loaves placed in a brine solution. The salted and/or brined curd pieces may then be placed in cheese hoops and may be pressed into blocks to form the cheese. The cheese may then be stored until the desired age is reached. In certain embodiments, the cheese may be stored in coolers. Finally, the cheese may be cut and packaged into blocks or other desired forms.

However, these processing steps are not without problem. For example, a common problem which occurs during bacterial fermentation processes, such as those involving lactic acid bacteria, is bacteriophage infection. In the dairy industry, the milk medium cannot be sterilized, and therefore complex dairy manufacturing processes may provide numerous opportunities for bacteriophage to build up and contaminate the milk.

Bacteriophage infection may lead to process inefficiencies, poor product quality, and in certain cases, failed fermentations. Many attempts have been made to solve this problem, such as by developing bacteriophage-resistant strains of bacteria. Common methods of developing these strains may include selection of naturally-occurring or induced mutants that are resistant to infection, transfer of naturally-occurring bacteriophage resistant mechanisms to sensitive strains, or the construction of new resistance mechanism using genetic engineering technologies. While these methods may slow the rate of phage infection, subsequent evolution may produce new phage that are not sensitive to the resistance mechanism. The result is that these strategies have failed to produce bacterial strains with long-term resistance to bacteriophage infection.

The fermentation industry has, therefore, been left with two options: either use multiple strains in a rotated pattern so that phage are unable to build up to levels high enough to impact the fermentation, or use complex strain mixtures so that if one strain is infected by bacteriophage it does not impact the fermentation. Both of these options, however, are less than ideal as they may each introduce unnecessary complexity and variability into the fermentation process.

Unlike bacteria, however, yeasts are not infected by viruses, and therefore, fermentations conducted with yeast strains may achieve consistent and efficient fermentations. This makes using yeast, rather than bacteria, for fermentation a means of solving the inconsistency and inefficiency of dairy manufacture that may result from bacteriophage infection.

The inventors have now discovered methods for acidifying milk, such as during the process of fermentation, which utilize yeast rather than bacteria, and which can be used in methods of making dairy products, such as cheese.

### SUMMARY

Disclosed herein are methods of acidifying milk comprising adding at least one microorganism belonging to the Kingdom Fungi to milk. According to certain exemplary embodiments, the milk may be acidified to a pH of less than about 6, such as a pH ranging from about 5.0 to about 5.6. Also disclosed herein are methods of making a dairy product, such as cheese, using at least one microorganism belonging to the Kingdom Fungi to acidify milk. Further disclosed herein are dairy products, such as cheese, made by acidifying milk using at least one microorganism belonging to the Kingdom Fungi.

In certain exemplary embodiments, the at least one microorganism belonging to the Kingdom Fungi may be chosen from the genera Kluyveromyces, Saccharomyces, and Candida, such as Kluyveromyces marxianus and Kluyveromyces lactis. In certain embodiments, the at least one microorganism belonging to the Kingdom Fungi may be provided in the form of frozen, liquid, and freeze-dried microorganisms. According to various exemplary embodiments, the at least one microorganism belonging to the Kingdom Fungi may be added to milk having a temperature ranging from about 25° C. to about 45° C., for a period of time of at least about 30 minutes. In certain other embodiments disclosed herein, rennet may be added to the milk after the milk has been acidified by the at least one microorganism belonging to the Kingdom Fungi. In further exemplary embodiments disclosed herein, at least one lactic acid bacteria may be added to the milk, such as, for example, those chosen from the genera Lactococcus, Streptococcus, and Lactobacillus. For example, the at least one lactic acid bacteria may be added to the milk by inoculating the milk through any inoculation method known in the art. In certain embodiments, the at least one lactic acid bacteria may be added to the milk in an inoculation percent ranging from about 0.0005% to about 0.001%. In various embodiments, the at least one microorganism belonging to the Kingdom Fungi is added to the milk in an inoculation percent ranging from about 0.1% to about 10%. As used herein, the term “inoculation percent” refers to the quantity of the organism added to milk, such that, for example, about 1% inoculation would equal about 1 pound of the inoculation organism added to about 100 pounds of milk.

### BRIEF DESCRIPTION OF THE DRAWINGS

FIG. 1 is a graph according to certain exemplary embodiments of the disclosure, illustrating the pH over time of milk inoculated with Strain Nos. 1-6 as shown in Table 1 below, as well as a whole milk control.

FIG. 2 is a graph according to certain exemplary embodiments of the disclosure, illustrating the pH over tune of milk inoculated with Strain Nos. 7-12 as shown in Table 1 below, as well as a whole milk control.

FIG. 3 is a graph according to certain exemplary embodiments of the disclosure, illustrating the pH over time of milk inoculated with Strain Nos. 13-24 as shown in Table 1 below, as well as a whole milk control.

FIG. 4 is a graph according to certain exemplary embodiments of the disclosure, illustrating the pH over time of milk inoculated with Strain Nos. 25-35 and 41 as shown in Table 1 below, as well as a whole milk control.

FIG. 5 is a graph according to certain exemplary embodiments of the disclosure, illustrating the pH over time of milk inoculated with Strain Nos. 34 and 36-40 as shown in Table 1 below.

FIG. 6 is a bar graph showing the acidification rate of Strain Nos. 1, 9, 13, 14, and 32 in 2% of a yeast growth media comprising 10% lactose, 1% yeast extract, and 2% peptone (hereinafter “YPL”), according to certain exemplary embodiments of the disclosure.

FIG. 7 is a bar graph showing the acidification rate of Strain Nos. 1, 9, 13, 14, and 32 in 2% of a yeast growth media comprising 2% lactose, 1% yeast extract, and 2% casein hydrolysate (hereinafter “YCL”), according to certain exemplary embodiments of the disclosure.

FIG. 8 is a bar graph showing the acidification rate of Strain Nos. 1, 9, 13, 14, and 32 in 10% YPL, according to certain exemplary embodiments of the disclosure.

FIG. 9 is a graph showing the optical density (hereinafter “OD”) at an absorbance of 600nm over time for Strain No. 14, which was grown in 2% YPL at an inoculation rate of 0.2 mL; 2% YPL at an inoculation rate of 0.8 mL; 10% YPL at an inoculation rate of 0.2 mL; and 10% YPL at an inoculation rate of 0.8 mL, according to certain exemplary embodiments of the disclosure.

FIG. 10 is a graph showing the OD at an absorbance of 600nm over time for Strain No. 32, which was grown in 2% YPL at an inoculation rate of 0.2 mL; 2% PYL at an inoculation rate of 0.8 mL; 10% YPL at an inoculation rate of 0.2 mL; and 10% YPL at an inoculation rate of 0.8 mL, according to certain exemplary embodiments of the disclosure.

FIG. 11 is a graph showing Strain 14 grown aerobically and anaerobically in 2% Yeast Extract Peptone Dextrose (hereinafter “YPD”) and inoculated in to 2% milk, 2% YPD, and 2% YPL, according to certain exemplary embodiments of the disclosure.

FIG. 12 is a graph showing the pH over time of ten bottles of 2% whole milk prepared with 3% methyl sulfonyl methane (hereinafter “MSM”) and/or 50 units of banana peel extract (hereinafter “BP”), according to certain exemplary embodiments of the disclosure.

### DESCRIPTION OF EXEMPLARY EMBODIMENTS

According to various embodiments of the disclosure, a microorganism chosen from the Kingdom Fungi may be added to milk to acidify the milk, such as during fermentation processes.

The fungi may serve to lower the pH of the milk by producing acid during the fermentation process. It may also contribute to the production of flavor compounds in the resultant dairy product, such as, for example, the cheese.

According to various embodiments, the fungi may be used in place of traditional lactic acid starter bacteria, or, in certain embodiments, as a supplement in combination with lactic acid bacteria, in the fermentation process, e.g. to produce a dairy product such as a cheese. In certain embodiments, a cheddar or gouda type cheese may be produced.

The fungi may be added to the mixture at any point during the process. By way of non-limiting example, the fungi may be added to the milk after a temperature adjustment (e.g. cooling) following pasteurization or a heating step. Optionally, the fungi may be added to the milk before a step of adding rennet. According to various embodiments of the disclosure, the fungi and optionally the lactic acid bacteria may be provided in one or more of frozen, liquid, and freeze-dried forms.

Non-limiting examples of fungi that may be chosen according to embodiments of the disclosure include, but are not limited to, fungi chosen from the genera Saccharomyces, Issatchenkia, Candida, and Kluyveromyces. In certain embodiments, for example, the fungi may be chosen from Kluyveromyces lactis, Kluyveromyces marxianus, Saccharomyces uvarum, Issatchenkia orientalis, Candida krusei, and Candida blankii. According to various non-limiting embodiments, the fungi may be chosen from Kluyveromyces marxianus and Kluyveromyces lactis

In certain embodiments disclosed herein, the at least one microorganism belonging to the Kingdom Fungi may be genetically modified so as to comprise, for example, heterologous genetic material. For example, in certain embodiments, the at least one microorganism belonging to the Kingdom Fungi may be genetically modified to enhance the microorganism's production of lactic acid. In certain exemplary embodiments, the at least one microorganism belonging to the Kingdom Fungi may be LDH+, meaning the genetic material of the microorganism codes for the expression of lactate dehydrogenase (LDH). In certain exemplary embodiments, the at least one microorganism belonging to the Kingdom Fungi may be PDC−, meaning the genetic material of the microorganism does not code for the expression of pyruvate decarboxylase (PDC). In certain embodiments, the at least one microorganism belonging to the Kingdom Fungi may be PDC+, meaning the genetic material of the microorganism codes for the expression of PDC. In various embodiments the at least one microorganism belonging to the Kingdom Fungi may be PDC reduced, meaning that the genetic material of the microorganism has been modified to reduce, but not eliminate, PDC activity. The microorganisms disclosed herein may be genetically modified by any method known to those skilled in the art.

According to various embodiments disclosed herein, the at least one microorganism belonging to the Kingdom Fungi may be added to the milk in an inoculation percent ranging up to about 15%, up to about 12%, or up to about 10%, such as from about 0.01% to about 15%, about 0.1% to about 10%, about 0.5% to about 8%, about 1% to about 5%, or about 2% to about 4%. For example, the at least one microorganism belonging to the Kingdom Fungi may be added to the milk in an inoculation percent of about 1%, about 2%, about 3%, about 4%, or about 5%.

In certain embodiments disclosed herein, at least one growth stimulator may optionally be added to increase the growth rate, acidification rate, and/or stability of the fungi. As used herein, the term “growth stimulator” is meant to include substances that, for example, may be electron acceptors, such as oxygen and other substances known in the art as growth stimulators. By way of non-limiting example, in certain embodiments, at least one growth stimulator chosen from yeast extract, nitrates, synthetic molecules, formate, pyruvate, methyl sulfonyl methane (MSM), and banana peel extract (BP) may be used.

The at least one microorganism chosen from the Kingdom Fungi may be added to milk by any process or method known to those of skill in the art, at any time. By way of non-limiting example, frozen inoculate may be added to the milk by way of direct vat inoculation. Alternatively, inoculate may be grown in a medium comprising milk and transferred to the milk to be inoculated by way of liquid inoculation. One skilled in the art would readily recognize appropriate inoculation methods that may be used. One skilled in the art would also recognize that the appropriate percentage of inoculate to be added would vary based on the method of inoculation. Although the embodiments disclosed herein and the percentages of inoculate refer to direct vat inoculation with frozen inoculate, it is also contemplated that liquid inoculation or any other inoculation method known in the art may also be used, with the percentage of inoculated appropriately adjusted to account for the inoculation method used.

According to at least certain exemplary embodiments, the at least one microorganism chosen from the Kingdom Fungi may be added to milk when the milk is at a temperature ranging from about 25° C. to about 45° C., such as from about 30° C. to about 38° C. or from about 30° C. to about 35° C. One skilled in the art would readily recognize that the temperature may vary depending on the microorganism added to the milk. For example, in certain embodiments, the at least one microorganism chosen from the Kingdom Fungi may be added to milk when the milk is at a temperature ranging from about 25° C. to about 30° C., about 30° C. to about 35° C., about 35° C. to about 40° C., and about 40° C. to about 45° C.

In certain embodiments, the fungi produce acid and thus lower the pH of the milk to a desired pH that is lower than the starting pH within a certain period of time, such as a period of time ranging up to about 30 minutes; a period of time of at least about 30 minutes, such as at least about 60 minutes; or a period of time ranging from about 30 minutes to about 60 minutes. In certain embodiments, the fungi may lower the pH of the milk to a pH of less than about 6, such as less than about 5.9, less than about 5.8, less than about 5.7, less than about 5.6, less than about 5.5, less than about 5.4, less than about 5.3, less than about 5.2, less than about 5.1, or less than about 5.0. In further embodiments, the pH of the milk may range from about 5.0 to about 5.6, or about 5.2 to about 5.4. In certain embodiments, the pH may be lowered to a desired pH in less than about 24 hours, such as less than about 10 hours, less than about 6.5 hours, less than about 5 hours, or less than about 4 hours.

In certain embodiments, in addition to the at least one microorganism chosen from the Kingdom Fungi, at least one starter culture, such as at least one lactic acid bacteria, may be added to the milk, The at least one lactic acid bacteria may be chosen, for example, from the genera Lactococcus, Streptococcus, and Lactobacillus. In certain embodiments, the at least one lactic acid bacteria may be chosen from Lactococcus lactis lactis, Lactococcus lactis cremoris, Streptococcus thermophiles, Lactobacillus delbruekii bulgaricus, Lactobacillus helveticus, Lactobacillus teasel, and Lactobacillus plantarum.

In certain exemplary embodiments disclosed herein, the at least one lactic acid bacteria may be added to the milk in an inoculation percent ranging from about 0.0005% to about 0.001%.

According to the methods for acidifying milk described herein, rennet may be added to the milk after it has been acidified by the at least one microorganism belonging to the Kingdom Fungi. In certain embodiments, the rennet is added to the milk after it reaches a pH of less than about 6.

Methods for making dairy products from milk disclosed herein may comprise, in addition to the acidification step using a microorganism belonging to the Kingdom Fungi, one or more steps chosen from pasteurization, heat treatment, inoculation with a starter culture, addition of rennet to the acidified milk, cutting, draining, milling, and/or shaping the curd, and storing, cutting and/or packaging the dairy product.

By way of non-limiting example, a method for making a dairy product comprising milk as disclosed herein comprises (a) optionally pasteurizing the milk, (b) adding at least one microorganism belonging to the Kingdom Fungi to the milk to produce acidified milk having a pH of less than about 6, (c) adding rennet to the acidified milk to produce curd, and (d) cutting, draining, milling, and/or shaping the curd to produce a dairy product.

Dairy products produced by the methods disclosed herein may include, for example, cheeses, such as cheddar cheese and gouda. Additional dairy products may be produced by the methods disclosed herein and can readily be envisioned by one skilled in the art without departing from the spirit and scope of the disclosure.

Unless otherwise indicated, all numbers used in the specification and claims are to be understood as being modified in all instances by the term “about,” whether or not so stated. It should also be understood that the precise numerical values used in the specification and claims form additional embodiments of the disclosure. Efforts have been made to ensure the accuracy of the numerical values disclosed in the Examples. Any measured numerical value, however, can inherently contain certain errors resulting from the standard deviation found in its respective measuring technique.

As used herein the use of “the,” “a,” or “an” means “at least one,” and should not be limited to “only one” unless explicitly indicated to the contrary.

It is to be understood that both the foregoing general description and the detailed description are exemplary and explanatory only and are not intended to be restrictive.

The accompanying drawings, which are incorporated in and constitute a part of this specification, are not intended to be restrictive, but rather illustrate embodiments of the disclosure.

Other embodiments will be apparent to those skilled in the art from consideration of the specification and practice of the disclosure.

### EXAMPLES

The following examples are illustrative only, are not intended to be limiting of the disclosure.

### Example 1.

Various strains of fungi, as shown in Table 1 below, prepared as below for further study.

TABLE 1

| Strain | | ATCC ID | |
|---|---|---|---|
| No. | Genus species | or Parent | Genotype* |
| 1, 40 | Kluyveromyces marxianus | 52486 | |
| 2 | Kluyveromyces marxianus | 52486 | PDC+, Lh-LDH+ (*) |
| 3 | Kluyveromyces marxianus | 52486 | PDC−, Lh-LDH+ |
| 4 | Kluyveromyces marxianus | 52486 | PDC− |
| 5 | Kluyveromyces lactis | 8585 | PDC−, LDH− |
| 6 | Kluyveromyces lactis | 8585 | PDC−, Bm-LDH+ (*) |
| 7 | Kluyveromyces marxianus | 52486 | PDC activity reduced |
| 8 | Kluyveromyces marxianus | 46537 | |
| 9 | Kluyveromyces lactis | 201343 | |
| 10 | Kluyveromyces marxianus | Wild isolate | |
| 11 | Candida blankii | 28532 | |
| 12 | Kluyveromyces marxianus | 60482 | |
| 13, 41 | Kluyveromyces lactis | 8585 | |
| 14 | Kluyveromyces lactis | 36940 | |
| 15 | Kluyveromyces lactis | 52733 | |
| 16 | Saccharomyces uvarum | 76514 | |
| 17 | Issatchenkia orientalis | Wild isolate | |
| 18 | Kluyveromyces marxianus | Wild isolate | |
| 19 | Kluyveromyces lactis | 36942 | |
| 20 | Kluyveromyces marxianus | Wild isolate | |
| 21 | Kluyveromyces lactis | 48789 | |
| 22 | Kluyveromyces marxianus | Wild isolate | |
| 23 | Kluyveromyces marxianus | Wild isolate | |
| 24 | Kluyveromyces marxianus | Wild isolate | |
| 25 | Kluyveromyces marxianus | Wild isolate | |
| 26 | Kluyveromyces marxianus | 64884 | |
| 27 | Candida krusei | Wild isolate | |
| 28 | Kluyveromyces marxianus | 48777 | |
| 29 | Kluyveromyces marxianus | 60480 | |
| 30 | Kluyveromyces marxianus | 36907 | |
| 31 | Kluyveromyces marxianus | 26548, NRRI, | |
| | | Y-7571 | |
| 32 | Kluyveromyces marxianus | Wild isolate | |
| 33 | Kluyveromyces marxianus | Wild isolate | |
| 34 | Kluyveromyces marxianus | Wild isolate | |
| 35 | Saccharomyces uvarum | 28097 | |
| 36 | Candida blankii | 18735 | |
| 37 | Kluyveromyces marxianus | Wild isolate | |
| 38 | Kluyveromyces marxianus | Wild isolate | |
| 39 | Kluyveromyces marxianus | Wild isolate | |

(*) Lactate dehydrogenase sourced from Lactobacillus helveticus (Lh) or Bacillus megatarium (Bm)

The fungi of strain numbers 1-41 were grown overnight in test tubes that were shaken at 30° C. and contained 10 nil of 2% YPD. Each strain was then added into bottles containing 100 g of 2% store bought milk at a rate of 0.5 ml/g. The milk bottles were incubated at 30° C., and the pH of the milk was followed using a Cinac for approximately 25 hours.

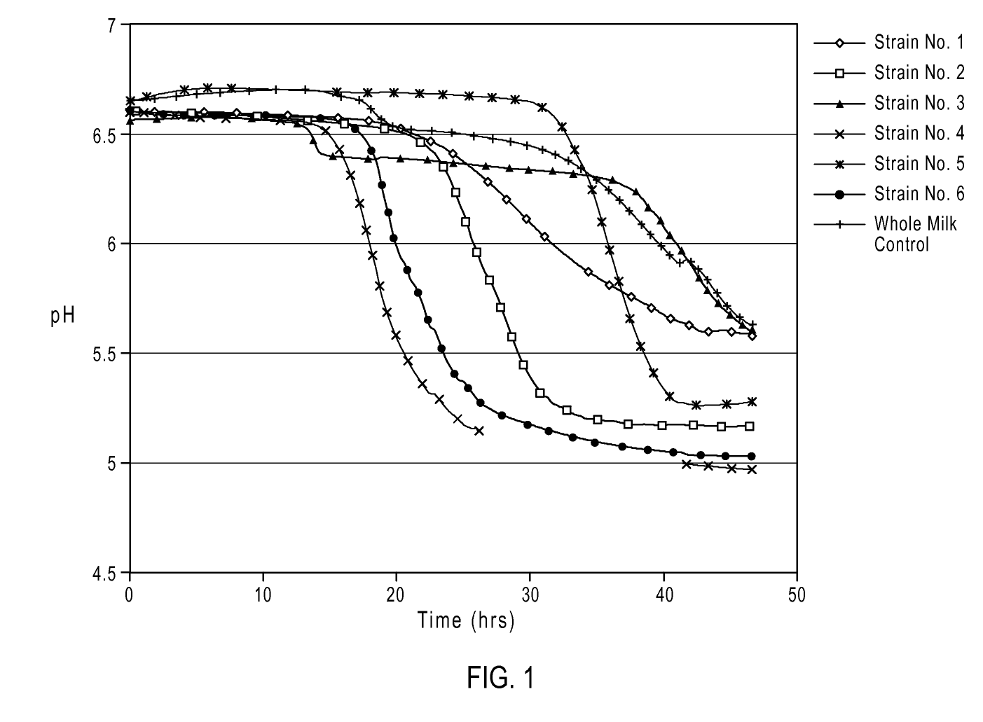

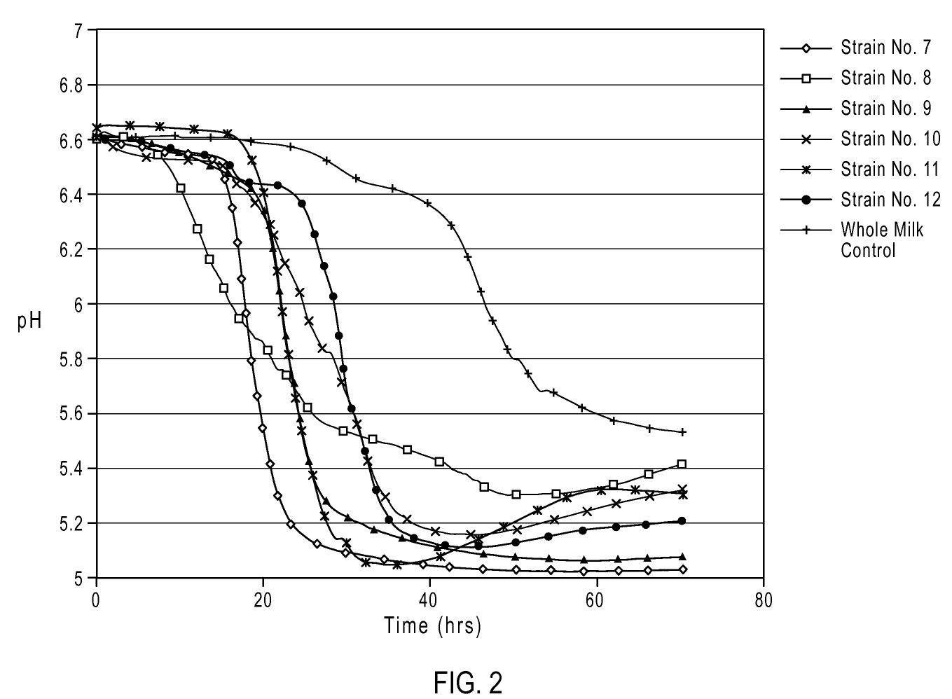

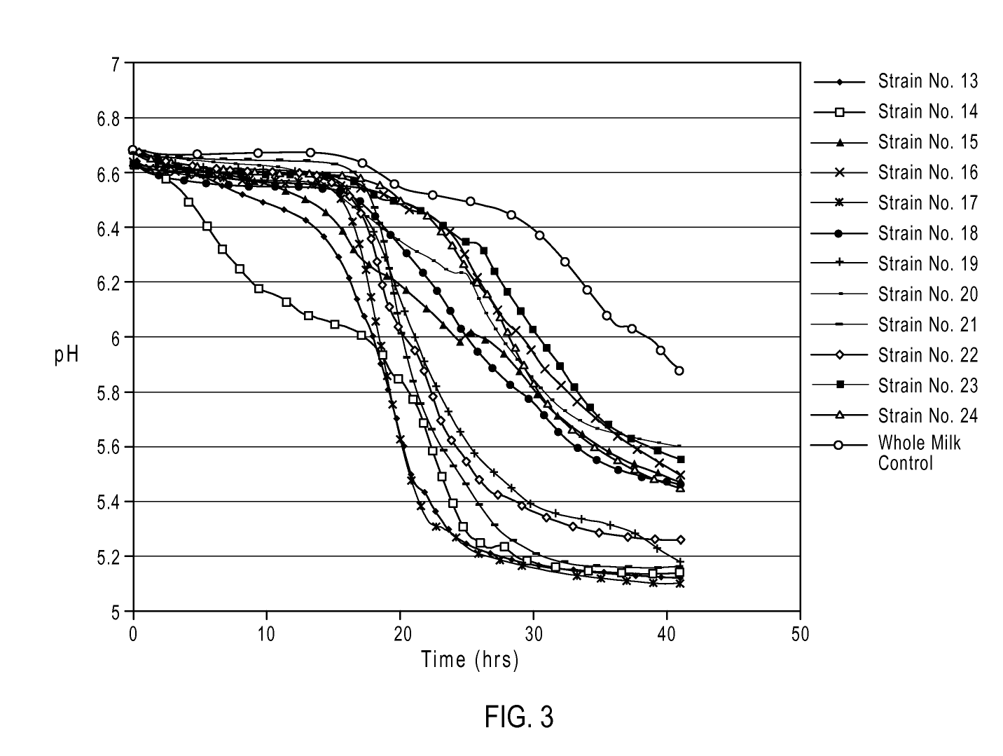

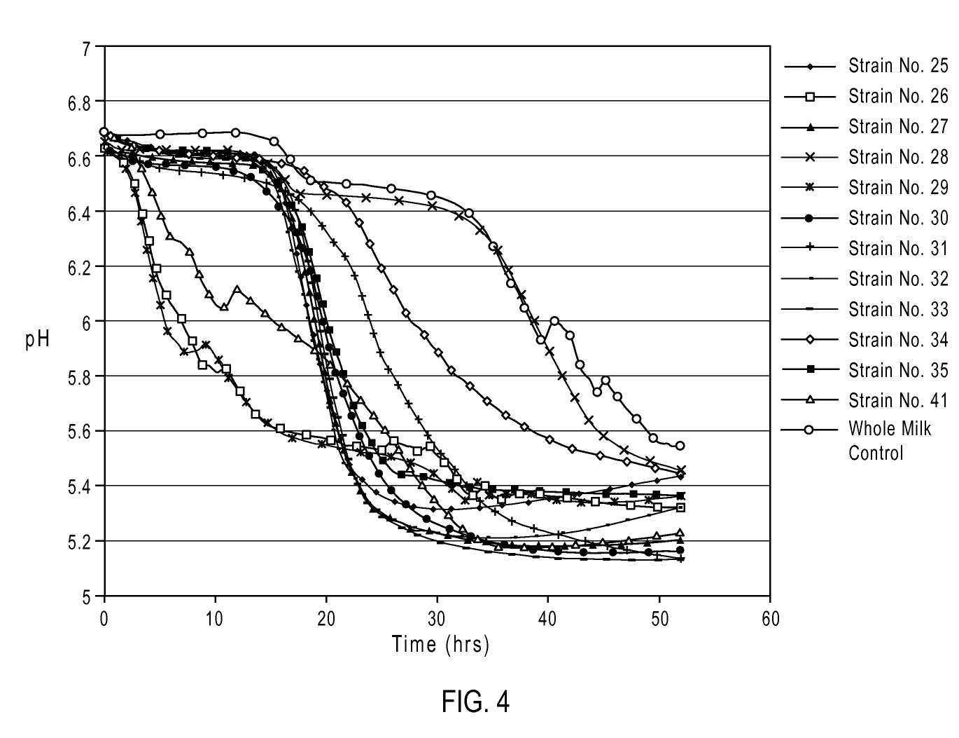

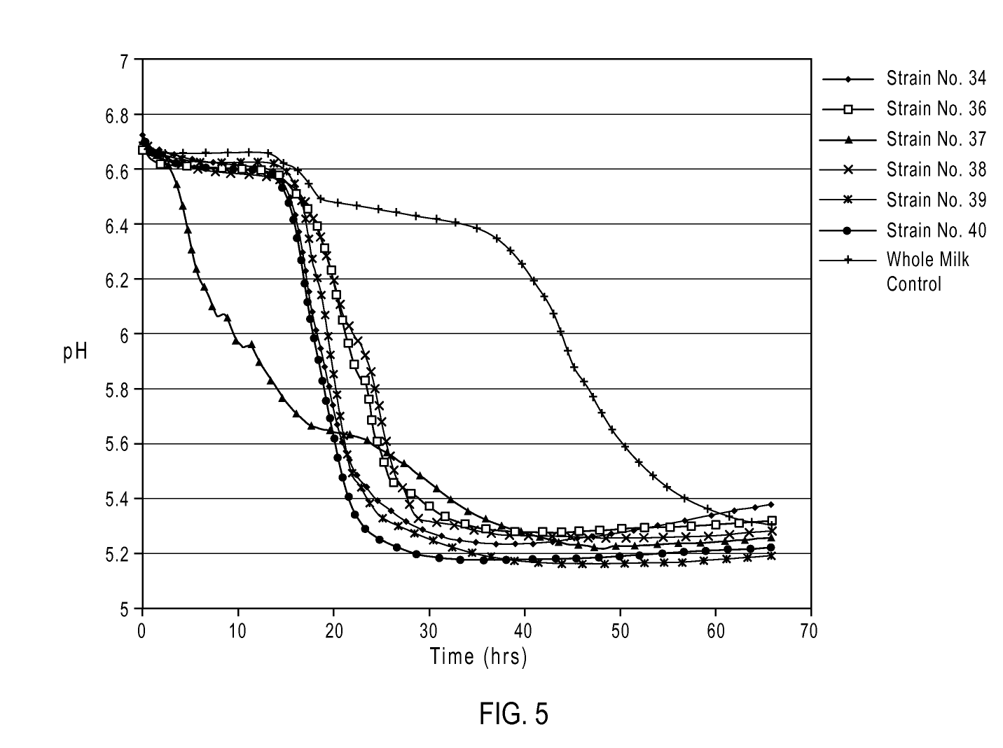

FIGS. 1-6 show the pH over time of the milk inoculated with the various strains of fungi.

### Example 2

Strain No. 37 (Kluyveromyces marxianus) was selected for further processing.

Strain No. 37 was transferred twice in 2% YPD broth and then grown overnight in 10 mL broth tubes at 30° C. with shaking at 300 rpm. The overnight culture was used at a rate of 30 mL in 3000 mL of 2% store bought milk, having an initial p11 of 6.62. The solution was heated to 86° C. for approximately 1 hour. The solution was diluted in 0.270 mL of cold water and cooled to a temperature of 30° C.

Two hours after inoculation, the pH was measured at 6.54, and the solution was cut randomly with a spatula slowly for approximately 5 minutes. The temperature was raised to 38° C., and the pH of the resultant curds, drained from the whey, was measured over time. Approximately 2 hours and 45 minutes after inoculation, the pH of the curds was 6.44. Approximately 3 hours and 15 minutes after inoculation, the pH of the curds was 6.35. Approximately 3 hours and 45 minutes after inoculation, the pH of the curds was 6.35. The solution was then left at room temperature overnight and pressed with beakers containing 1000 mL of water. Salt was added, and the pH of the curds was measured at 6.10.

### Example 3

Strain Nos. 1, 9, 13, 14, and 32 were selected to test the effects of different sugar levels and protein sources on the acidification rate.

Each of the five strains was grown on the following three media:
(1) 10% YPL yeast growth media (comprising 10% lactose, 1% yeast extract, and 2% peptone);
(2) 2% YPL yeast growth media (comprising 2% lactose, 1% yeast extract, and 2% peptone); and
(3) 2% YCL yeast growth media (comprising 2% lactose, 1% yeast extract, and 2% casein hydrolysate).

The strains were grown in test tubes containing 10 mL of each media that had been incubated statically at 30° C. for 48 hours. Tubes were inoculated with 0.2 mL of the respective 48 hour old cultures. Two sets of experiments were run. The pH of each culture was determined initially and again after 5.5 hours, 23 hours, and 30 hours.

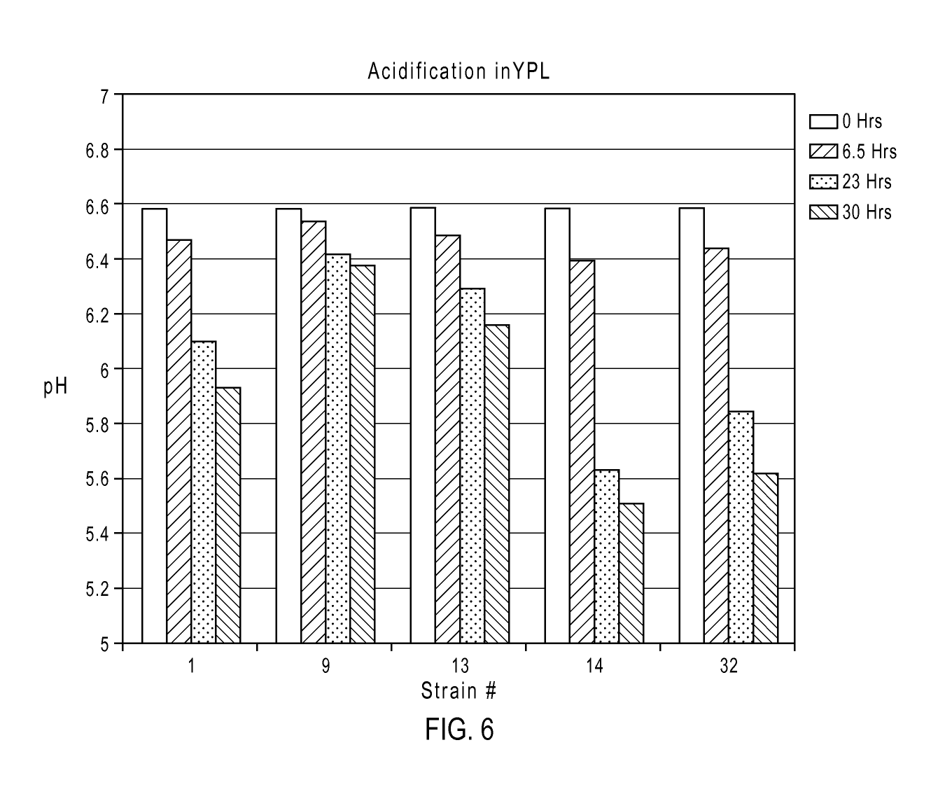

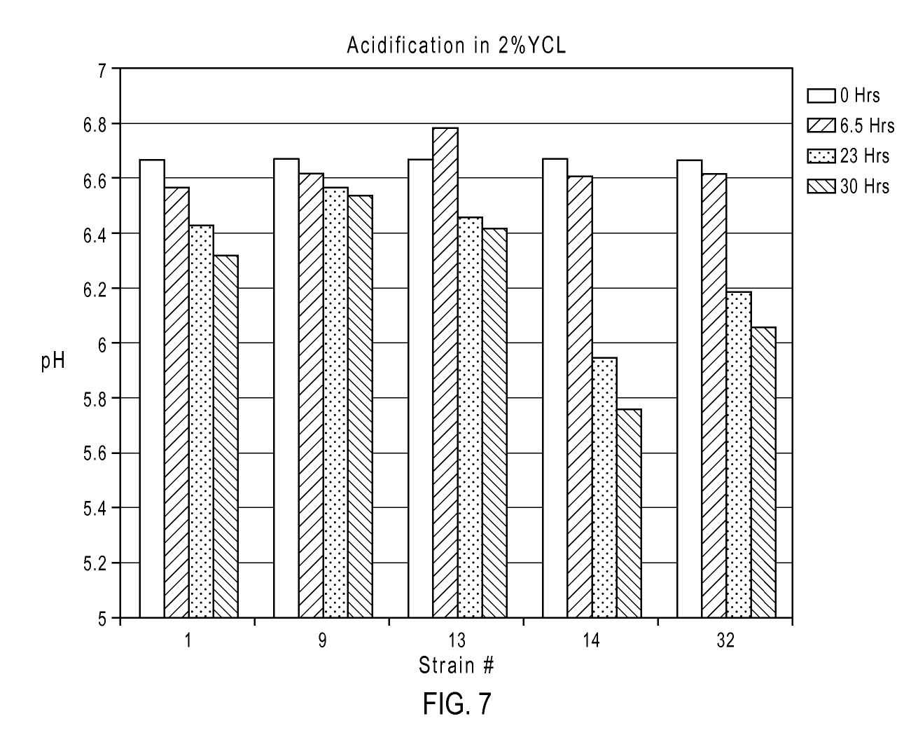

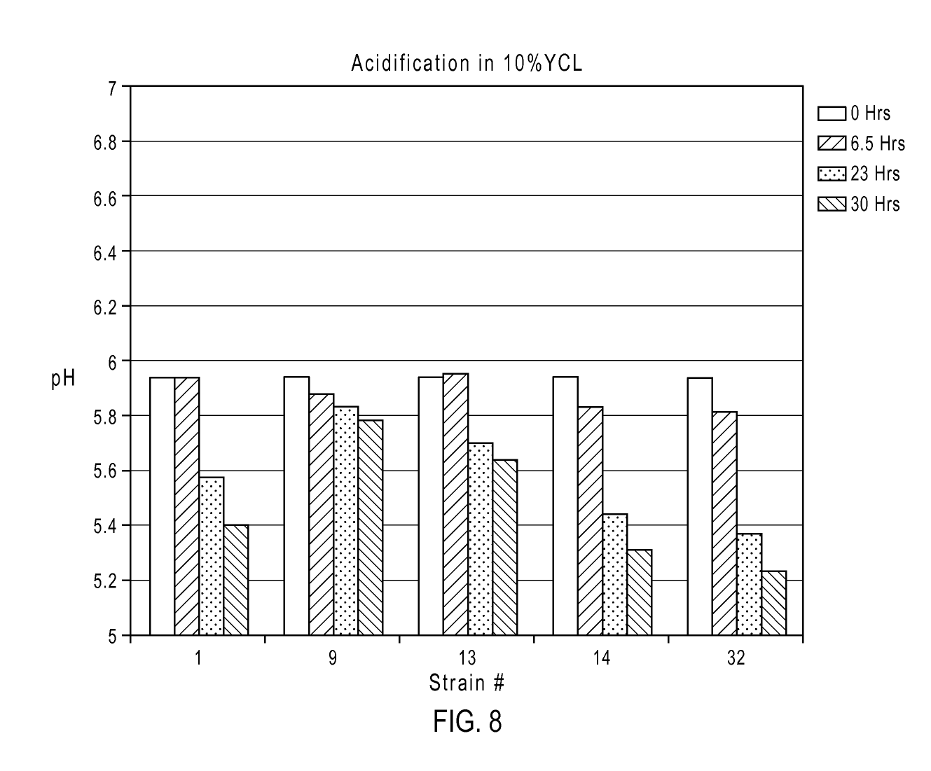

FIG. 6 shows the acidification rate of the five tested strains in 2% YPL at the various time points. FIG. 7 shows the acidification rate of the five tested strains in 2% YCL at the various time points, and FIG. 8 shows the acidification rate of the five tested strains in 10% YPL.

The results show that the rate of acid production may be most impacted by the lactose concentration, with the high concentration of lactose resulting in faster acid production. This is consistent with lactose transport being a rate limiting step in acidification. The results further show that casein is not an optimal nitrogen source, as the rate of acid production was better on peptone than on casein. Without wishing to be bound by theory, it is believed that this is not due to an inability to hydrolyze casein, as the casein used had been pre-hydrolyzed.

### Example 4

It is also theorized that one reason for the slow rate of acid production may be the long lag phase of the yeast. Thus, the following experiments were conducted to determine if the lag phase could be reduced by increasing the inoculation rate. Strain Nos. 7, 14, and 32 were selected.

Strain Nos. 14 and 32 were inoculated into 10 mL test tubes containing either 2% YPL or 10% YPL and incubated statically at 30° C. for approximately 16 hours. Strain Nos. 14 and 32 were then inoculated into 250 mL flasks containing either 75 mL of 2% YPL or 10% YPL. The flasks were inoculated at 2 different inoculation rates (0.2 mL and 0.8 mL) and incubated statically at 30° C. After inoculation, the absorbance at 600 nm was periodically determined by removing approximately 5 mL aliquots from each flask and using the aliquot to obtain the optical density (OD) using a Spec 20D+ spectrophotometer.

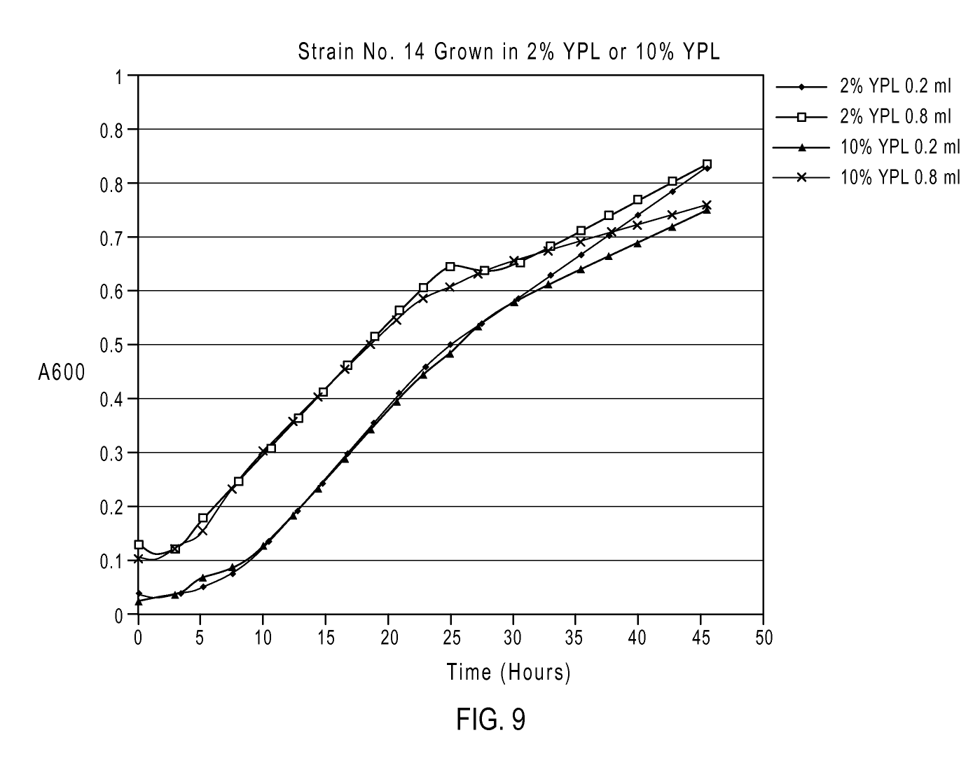

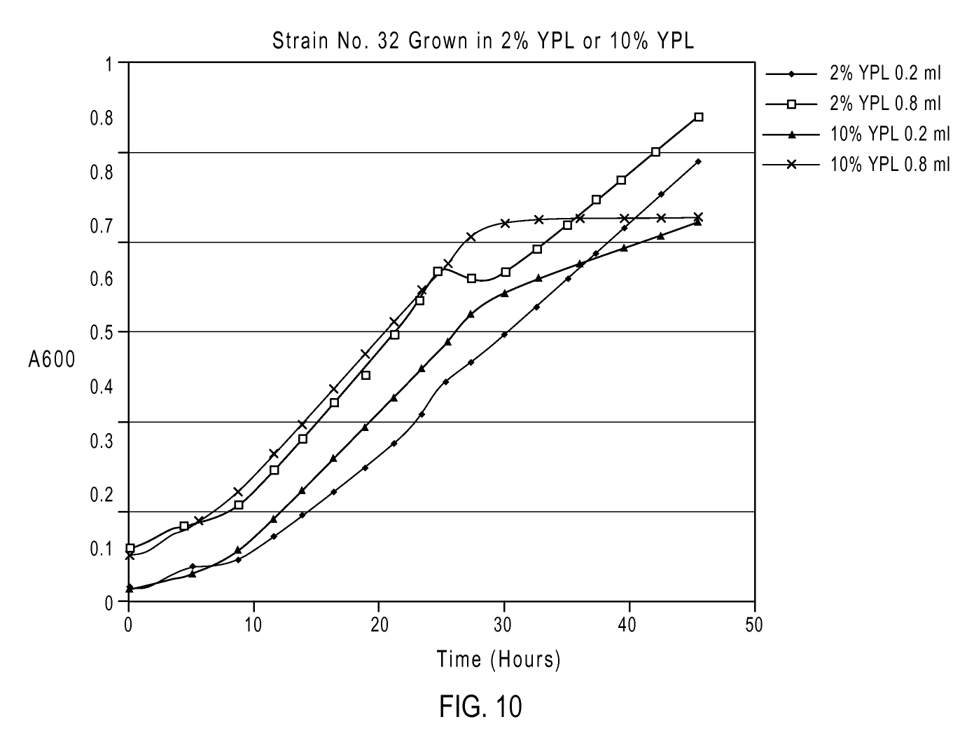

The results for Strain 14 are shown in FIG. 9, and the results for Strain 32 are shown in FIG. 10. The results indicate that Strain 14 appeared to have a shorter lag and faster initial growth rate at the higher inoculation rate. In contrast to the acidification rate, which varied greatly with sugar concentration, the growth rate was the same or very close regardless of the sugar concentration. Therefore, the rate of acid production appears to be disproportionately high compared to the growth rate at high sugar concentrations.

### Example 5

An experiment was conducted to determine the effect of growing inoculums under aerobic conditions before adding them to milk, in order to determine the acidification profiles. Strain Nos. 7, 14, and 32 were grown overnight in Belco 300 mL flasks with 4 bottom baffles. Each flask contained 75 mL of 2% YPL and was incubated at 30° C. in a shaker set at 300 rpm. The inoculums for the shake flasks were grown in test tubes containing 10 mL of 2% YPL. The tubes were incubated for 48 hours at 30° C. The 300 mL flasks were inoculated with 0.3 mL from the respective 48 hour old cultures of the three strains.

After overnight growth in the shake flasks, the OD at an absorbance of 600 nm was determined for each strain. Again, the inoculums were adjusted such that a 1× concentration contained 1 mL of culture at an OD of A600 of 0.300. Concentration rates of 1×, 5×, and 10× were used. 100 g bottles of ultra-heat treatment (UHT) milk were tempered to 30° C. and inoculated as described. The acidification rate of each bottle was determined using a Cinac and measured for 24 hours.

The results demonstrated that, for Strain 14, the initial rate of acid production increased in increasing inoculation rate, with the highest inoculation rate (10×) being the first to reach a pH of 6 at approximately 12 hours. However, once out of lag phase, the rate of acid production varied inversely with inoculation rate, with the result that all three inoculation rates for Strain 14 finished at the same pH of 5.62 after 24 hours.

An opposite pattern was observed for Strains 7 and 32. The 10× (or 5× for Strain 7) inoculation rate was the slowest to acidify, and the 1× inoculation rate was the fastest to acidify. The 1× inoculation achieved nearly the same pH at the same time as the Strain 14 cultures. It was also noted that the control, containing no added culture, did begin to acidify after about 17 to 18 hours. Therefore, the control results show that the rate of acidification for all bottles should be adjusted accordingly.

### Example 6

An experiment was conducted with Strain 14, as this strain exhibited the shortest lag phase, demonstrated in Example 5 above. The experiment directly compared inoculums that were grown aerobically in flasks versus those grown statically in test tubes. Also, the strain was inoculated into 2% YPL and 2% YPD bottles in addition to bottles containing 2% store bought milk to determine if the long lag time could be attributed solely to milk as a substrate.

Strain 14 was grown overnight in a 300 mL shake flask containing 75 mL of 2% YPD and in test tubes containing 10 mL of 2% YPD. Both cultures were incubated at 30° C. The 300 mL flask was placed in a shaker set to 300 rpm, while the test tube cultures were incubated statically. The following morning, the OD A600 for each culture was determined. As previously, culture inoculums were adjusted based on a 1× concentration being equal to 1 mL at an OD A600 of 0.300. A 5× inoculation rate was used throughout.

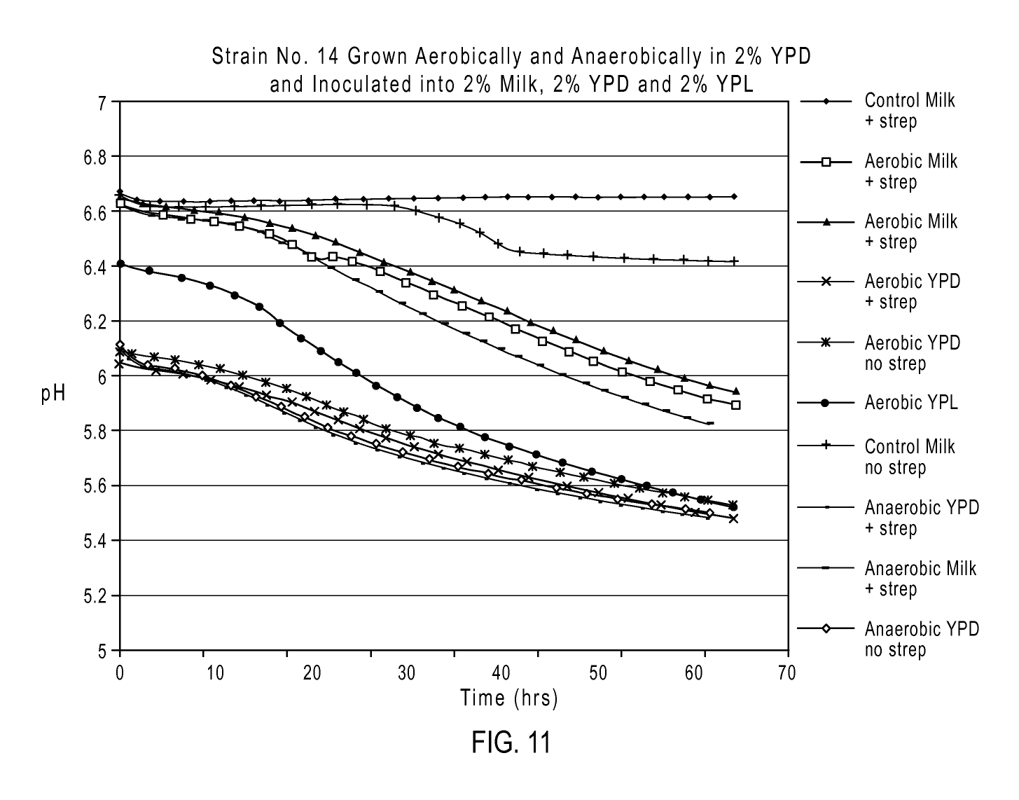

In order to eliminate the acidification of the control milk as a variable, streptomycin (1 ug/g medium) was added to some test bottles to suppress the native bacterial flora. Bottles containing either 100 g of 2% YPD, 2% YPL, or 2% whole milk were tempered to 30° C. and inoculated with either 5× concentration of Strain 14 grown aerobically or a 5× concentration of Strain 14 grown statically. The acidification rate was followed in each bottle using a Cinac. The acidification rate was monitored over approximately 24 hours. The results are show in FIG. 11.

The results illustrate that acid production over the first five hours was approximately the same regardless of medium and whether the inoculums were grown aerobically or anaerobically. However, over 24 hours, the rate of acid production in milk was slightly faster for cells grown anaerobically. This suggests that pre-growth in 2% YPD and transfer to 2% YPL results in a slightly faster rate of acid production. Results are also consistent, demonstrating that inoculums grown anaerobically are faster acidifying than those grown aerobically.

### Example 7

Strain 14 was grown in 300 mL shake flasks containing 2% YPL and either methyl sulfonyl methane (MSM) or banana peel extract (BP). These cultures were inoculated with 0.13 mL of a 48-hour-old culture of Strain 14 grown in 10 mL of 2% YPL. The culture flasks were incubated overnight at 30° C. at 300 rpm. The following morning, the OD A600 was determined for each culture and inoculation levels were adjusted to contain a 5× rate (1×=1 mL of OD A600 culture at 0.300).

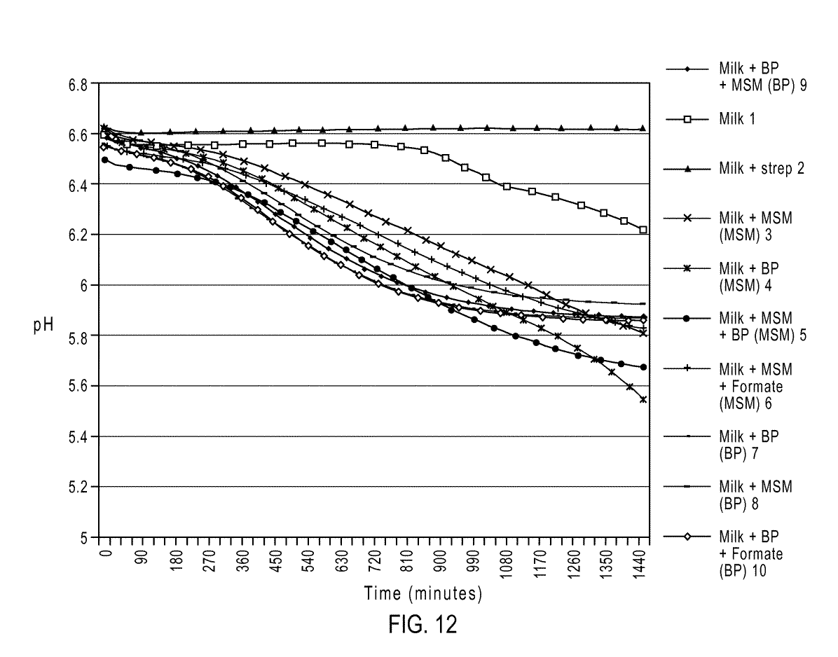

Ten bottles containing 100 g of 2% whole milk were prepared and 3% MSM and/or 50 units of BP were added to 8 bottles. Two bottles also contained 0.002% formate. All but one control bottle contained 100 μg of streptomycin. The bottles were tempered at 30° C., and the acidification pattern in each bottle was measured using a Cinac. The run lasted 24 hours. Inoculum grown in the shake flask containing 3% MSM is designated in FIG. 12 by “(MSM)”, and likewise inoculum grown in the shake flask containing 50 units of BP is designated in FIG. 12 by “(BP)”.

The results show that the milk bottles containing BP and BP plus formate and grown in the shake flask containing 50 units of BP were the fastest to acidify to a pH of 6. The bottle containing 3% MSM and 50 units of BP and grown in the shake flask containing 3% MSM was the fastest to reach a pH of 5.8, and the bottle containing 50 units of BP and grown in the presence of 3% MSM was the only bottle to reach a pH of 5.6 in 24 hours. It was also the only bottle that looked like acid production was accelerated rather than slowing or remaining steady at the end of the 24 hours. The results suggest that the presence of either MSM and/or BP may have a positive effect on acid production and potentially the lag time of the yeast.

It is noted that, in this experiment, the shake flask containing the 50 units of BP grew to an OD A600 level twice as great as the shake flask containing 3% MSM. Both shake flasks received the same amount of inoculums (0.13 mL) from a test tube of Strain 14 grown overnight in 10 mL 2% YPL. An aliquot taken from the BP shake flask and examined under a microscope did not exhibit any contamination. Thus, contamination could not account for the differences in cell growth observed using these two compounds. The data supports the observation that Strain 14 grown aerobically in 2% YPL containing 50 units of BP grew to twice the cell number in the same time as compared to Strain 14 supplemented with 3% MSM. This indicates that BP may be used as a growth stimulator for Strain 14 or other yeast strains of the genus Kluyveromyces.

1. A method for acidifying milk comprising:
adding at least one microorganism belonging to Kingdom Fungi to milk,
wherein the milk is acidified by the at least one microorganism belonging to the Kingdom Fungi to a pH of less than about 6.

2. The method for acidifying milk according to claim 1, wherein the at least one microorganism belonging to the Kingdom Fungi is chosen from genera Kluyveromyces, Issatchenkia, Saccharomyces, and Candida.

3. The method for acidifying milk according to claim 1, or wherein the at least one microorganism belonging to the Kingdom Fungi is from genus Kluyveromyces and is chosen from species marxianus and lactis.

4. The method for acidifying milk according to claim 1, wherein the milk is acidified to a pH ranging from about 5.0 to about 5.6.

5. The method of acidifying milk according to claim 1, wherein the at least one microorganism belonging to the Kingdom Fungi is chosen from frozen, liquid, and freeze-dried forms.

6. The method of acidifying milk according to claim 1, wherein the at least one microorganism belonging to the Kingdom Fungi is added to milk having a temperature ranging from about 25° C. to about 45° C., for a period of time of at least about 30 minutes.

7. The method of acidifying milk according to claim 1, further comprising adding rennet to the milk after the milk is acidified by the at least one microorganism belonging to the Kingdom Fungi to a pH of less than about 6.

8. The method of acidifying milk according to claim 1, further comprising adding at least one lactic acid bacteria chosen from genera Lactococcus, Streptococcus, and Lactobacillus to the milk.

9. The method of acidifying milk according to claim 8, wherein the at least one lactic acid bacteria is added to the milk in an inoculation percent ranging from about 0.0005% to about 0.001%.

10. The method of acidifying milk according to claim 1, wherein the at least one microorganism belonging to the Kingdom Fungi is added in an inoculation percent ranging from about 0.01% to about

11. The method of acidifying milk according claim 1, wherein the at least one microorganism belonging to the Kingdom Fungi is added in an inoculation percent ranging from about 0.1% to about 10%.

12. A method for making a dairy product comprising milk, the method comprising:
(a) optionally pasteurizing the milk,
(b) adding at least one microorganism belonging to Kingdom Fungi to the milk to produce acidified milk having a pH of less than about 6,
(c) adding rennet to the acidified milk to produce curd, and
(d) cutting, draining, milling, and/or shaping the curd to produce a dairy product.

13. The method for making a dairy product according to claim 12, wherein the at least one microorganism belonging to the Kingdom Fungi is chosen from genera Kluyveromyces, Issatchenkia, Saccharomyces, and Candida.

14. The method for making a dairy product according to claim 12, wherein the at least one microorganism belonging to the Kingdom Fungi is from genus Kluyveromyces and is chosen from species marxianus and lactis.

15. The method for making a dairy product according to claim 12, wherein the acidified milk has a pH ranging from about 5.0 to about 5.6.

16. The method of making a dairy product according to any claim 12, wherein the at least one microorganism belonging to the Kingdom Fungi is chosen from frozen, liquid, and freeze-dried forms.

17. The method of making a dairy product according to any claim 12, wherein the at least one microorganism belonging to the Kingdom Fungi is added to milk having a temperature ranging from about 25° C. to about 45° C., for a period of time of at least about 30 minutes.

18. The method of making a dairy product according to claim 12, further comprising adding at least one lactic acid bacteria chosen from genera Lactococcus, Streptococcus, and Lactobacillus to the milk.

19. The method of making a dairy product according to claim 18, wherein the at least one lactic acid bacteria is added to the milk in an inoculation percent ranging from about 0.0005% to about 0.001%,

20. The method of making a dairy product according to claim 11, wherein the at least one microorganism belonging to the Kingdom Fungi is added in an inoculation percent ranging from about 0.01% to about 15%.

21. The method of making a dairy product according to claim 12, wherein the at least one microorganism belonging to the Kingdom Fungi is added in an inoculation percent ranging from about 0.1% to about 10%.

22. A dairy product comprising milk produced according to the method in claim 12.

23. The dairy product according to claim 22, herein the dairy product is a cheese.
